(19) United States
(12) Patent Application Publication
(10) Pub. No.: US 2026/0290522 A1
(43) Pub. Date: Sep. 24, 2026

(54) METHODS AND SYSTEMS FOR SECURED CLOUD-BASED PROCESSING AND STORAGE BY DISTRIBUTED ALLOCATION

(71) Applicant: KONINKLIJKE PHILIPS N.V., EINDHOVEN (NL)

(72) Inventor: ANDRÉ FRANK SALOMON, AACHEN (DE)

(21) Appl. No.: 19/473,191

(22) PCT Filed: Apr. 11, 2024
(86) PCT No.: PCT/EP2024/059761

§ 371 Date: Oct. 7, 2025

Related U.S. Application Data
(60) Provisional application No. 63/459,277, filed on Apr. 14, 2023.

(51) Int. Cl. G16H 10/60; G06F 21/60; G06F 21/62

(52) U.S. Cl. CPC G16H 10/60; G06F 21/602; G06F 21/6254

(57) ABSTRACT

A method (100) for processing secure clinical data with a secure data processing system (200) comprising a local data center (270) and a plurality of distributed processors (280), comprising: receiving (120) secure clinical data associated with a patient and a processing request for the clinical data; determining (130) a segmentation protocol for the received secure clinical data; segmenting (140) the received secure clinical data based on the determined segmentation protocol to generate a plurality of clinical data segments; distributing (150) the plurality of clinical data segments to the plurality of distributed processors; receiving (160) a plurality of processed clinical data segments from the plurality of distributed processors; combining (170) the received plurality of processed clinical data segments to generate a single combined processed secure clinical data result.

### FIELD OF THE DISCLOSURE

The present disclosure is directed generally to methods and systems for processing and storing secure clinical data with a secure data processing system and distributed allocation.

### BACKGROUND

Data security is a constant concern. In the clinical setting, data security is often mandated by regulatory and ethical requirements. The hardware and maintenance infrastructure installed in the clinical setting generates significant additional costs, and increases the risk of data loss or leak. Complex processing and secured data storage systems, such as those in precision diagnostic devices like CT and MR have become a significant part of the clinical IT infrastructure which must be serviced by expensive third-party services or vendors.

As an alternative, low-cost and flexible computation power is offered by cloud-based services which may help to outsource some of the risks and potential issues for the clinical setting. However, one of the key limitations of cloud-based processing and storage is the potential risk of data leakage and misuse, which is a huge liability within the clinical environment. Certainly one major concern is that a third-party's security system fails (e.g., a cloud-based service is hacked) leading to critical data leakage of patient data.

Therefore, skepticism is widespread within the clinical setting regarding external cloud-based processing and storage of clinical data, although remote processing offers numerous advantages over classical on-site computing and infrastructure maintenance, which is needed for several tasks such as e.g. data reconstruction and image post-processing. Indeed, hardware maintenance, software costs, and downtime are costly and inefficient.

### SUMMARY OF THE DISCLOSURE

Accordingly, there is a continued need for improved methods and systems that can securely process and/or store secure clinical data within a distributed data processing and storage system. Various embodiments and implementations herein are directed to methods and systems for processing and storing secure clinical data with a secure data processing system and distributed allocation. The secure data system comprises a local data center and a plurality of distributed processors. The system receives secure clinical data associated with a patient and a processing request for the clinical data, and determines, based on the received processing request, a segmentation protocol for the received secure clinical data. The received secure clinical data is segmented based on the determined segmentation protocol to generate a plurality of clinical data segments. These clinical data segments are then distributed to the distributed processors, which perform the requested processing. The local data center then receives the processed clinical data segments in return, and combines the received clinical data segments to generate a single combined processed secure clinical data result.

Generally, in one aspect, a method for processing secure clinical data with a secure data processing system comprising a local data center and a plurality of distributed processors is provided. The method includes: (i) receiving, by the local data center, secure clinical data associated with a patient and a processing request for the clinical data; (ii) determining, based on the received processing request, a segmentation protocol for the received secure clinical data; (iii) segmenting the received secure clinical data based on the determined segmentation protocol to generate a plurality of clinical data segments; (iv) distributing the plurality of clinical data segments to the plurality of distributed processors, wherein a distributed processor performs the requested processing of a distributed clinical data segment to generate a processed clinical data segment; (v) receiving, by the local data center, a plurality of processed clinical data segments from the plurality of distributed processors; and (vi) combining the received plurality of processed clinical data segments to generate a single combined processed secure clinical data result.

In accordance with an embodiment, the clinical data is imaging data obtained by an imaging modality, and wherein the imaging modality is the local data center or is in communication with the local data center.

In accordance with an embodiment, the method further includes encrypting some or all of the plurality of clinical data segments.

In accordance with an embodiment, the plurality of clinical data segments are segmented such that a clinical data segment sent to a distributed processor cannot identify the patient.

In accordance with an embodiment, the method further includes providing, via a user interface, the single combined processed secure clinical data result.

In accordance with an embodiment, the method further includes diagnosing the patient based on the single combined processed secure clinical data result; and administering a treatment based on the diagnosis.

According to another aspect is a method for archiving secure clinical data with a secure data processing system comprising a local data center and a plurality of distributed databases. The method includes: (i) receiving, by the local data center, secure clinical data associated with a patient and an archiving request for the clinical data; (ii) determining, based on the received archiving request, a segmentation protocol for the received secure clinical data; (iii) encrypting the received secure clinical data to generate encrypted secure clinical data; (iv) segmenting the received encrypted secure clinical data based on the determined segmentation protocol to generate a plurality of encrypted clinical data segments; (v) replicating some or all of the plurality of encrypted clinical data segments to generate redundancy of the secure clinical data; and (vi) distributing the plurality of encrypted clinical data segments to the plurality of distributed databases for storage.

In accordance with an embodiment, the method further includes receiving, by the local data center in response to a received request for the secure clinical data, a plurality of encrypted clinical data segments from the plurality of distributed databases; decrypting the received plurality of encrypted clinical data segments to generate a plurality of decrypted clinical data segments; and combining the plurality of decrypted clinical data segments to generate a single combined secure clinical data result.

In accordance with an embodiment, the local data center maintains a record of which distributed databases store which encrypted clinical data segments.

According to another aspect is a secure data processing system for processing or storing secure clinical data. The system includes a local data center in communication with a plurality of distributed processors and/or a plurality of distributed processors; a processor configured to: (i) receive both (a) a secure clinical data associated with a patient and (b) a processing request or a storage request for the clinical data; (ii) determine, based on the received processing request or storage request, a segmentation protocol for the received secure clinical data; (iii) segment the received secure clinical data based on the determined segmentation protocol to generate a plurality of clinical data segments; (iv) distribute the plurality of clinical data segments to the plurality distributed processors and/or to the plurality of distributed processors.

In accordance with an embodiment, the processor is further configured to encrypt some or all of the plurality of clinical data segments.

In accordance with an embodiment, the processor is further configured to: (v) receive a plurality of processed clinical data segments from the plurality of distributed processors; and combine the received plurality of processed clinical data segments to generate a single combined processed secure clinical data result.

In accordance with an embodiment, the processor is further configured to provide, via a user interface, the single combined processed secure clinical data result.

In accordance with an embodiment, the processor is further configured to replicate some or all of the plurality of encrypted clinical data segments to generate redundancy of the secure clinical data.

In accordance with an embodiment, the processor is further configured to: (v) receive, in response to a received request for the secure clinical data, a plurality of encrypted clinical data segments from the plurality of distributed databases; (vi) decrypt the received plurality of encrypted clinical data segments to generate a plurality of decrypted clinical data segments; and (vii) combine the plurality of decrypted clinical data segments to generate a single combined secure clinical data result.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for processing secure clinical data, in accordance with an embodiment.

FIG. 2 is a schematic representation of a secure data processing system, in accordance with an embodiment.

FIG. 3 is a schematic representation of a system for processing secure clinical data, in accordance with an embodiment.

FIG. 4 is a flowchart of a method for storing secure clinical data, in accordance with an embodiment.

FIG. 5 is a schematic representation of a system for storing secure clinical data, in accordance with an embodiment.

### DETAILED DESCRIPTION OF EMBODIMENTS

The present disclosure describes various embodiments of a system and method configured to safely and securely process or store sensitive clinical data. More generally, Applicant has recognized and appreciated that it would be beneficial to provide secure data processing and storage. Accordingly, a secure data system comprises a local data center and a plurality of distributed processors. The system receives secure clinical data associated with a patient and a processing request for the clinical data, and determines, based on the received processing request, a segmentation protocol for the received secure clinical data. The received secure clinical data is segmented based on the determined segmentation protocol to generate a plurality of clinical data segments. These clinical data segments are then distributed to the distributed processors, which perform the requested processing.

According to an embodiment, the methods and systems described or otherwise envisioned herein provides numerous advantages over existing methods. For example, the secure data systems and methods described or otherwise envisioned herein ensures the security of sensitive clinical data. The odds of a number of different data leakages happening at different allocation locations, in parallel allowing the decryption and combination of allocated data segments, is very small. The proposed secure data systems and methods described or otherwise envisioned herein also reduce costs associated with internal processing and storage, including both hardware and software installation and maintenance costs.

The embodiments and implementations disclosed or otherwise envisioned herein can be utilized with any clinical setting or system. For example, one application of the embodiments and implementations herein is to improve analysis systems such as, e.g., the Philips® IntelliSpace® product (manufactured by Koninklijke Philips, N.V.), among many other products. However, the disclosure is not limited to these devices or systems, and thus the disclosure and embodiments disclosed herein can encompass any device or system capable of creating or handling secure clinical data.

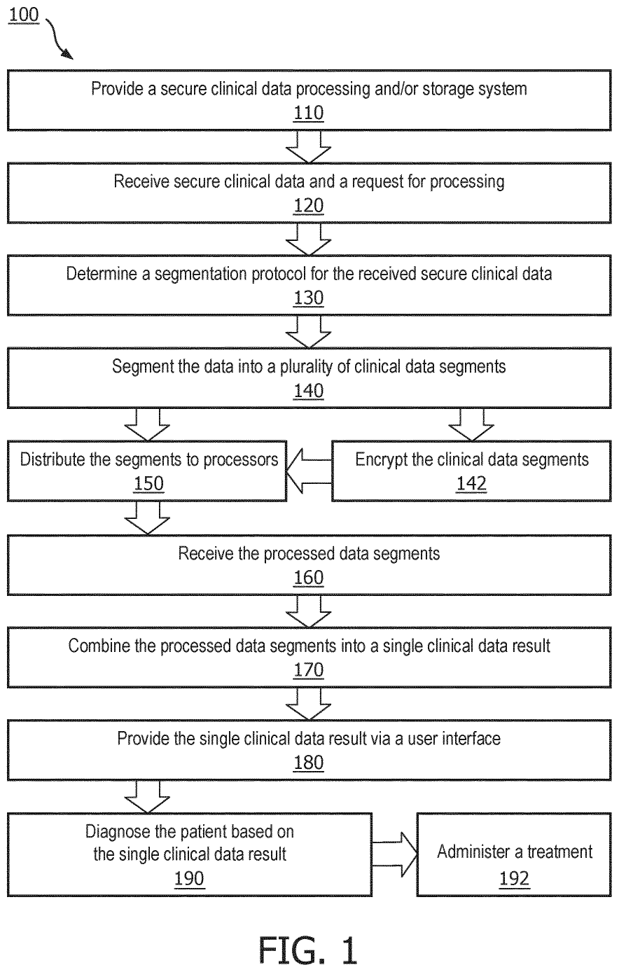

Referring to FIG. 1, in one embodiment, is a flowchart of a method 100 for processing secure clinical data with a secure data processing system. The methods described in connection with the figures are provided as examples only, and shall be understood not to limit the scope of the disclosure. The secure data processing system can be any of the systems described or otherwise envisioned herein. The secure data processing system can be a single system or multiple different systems.

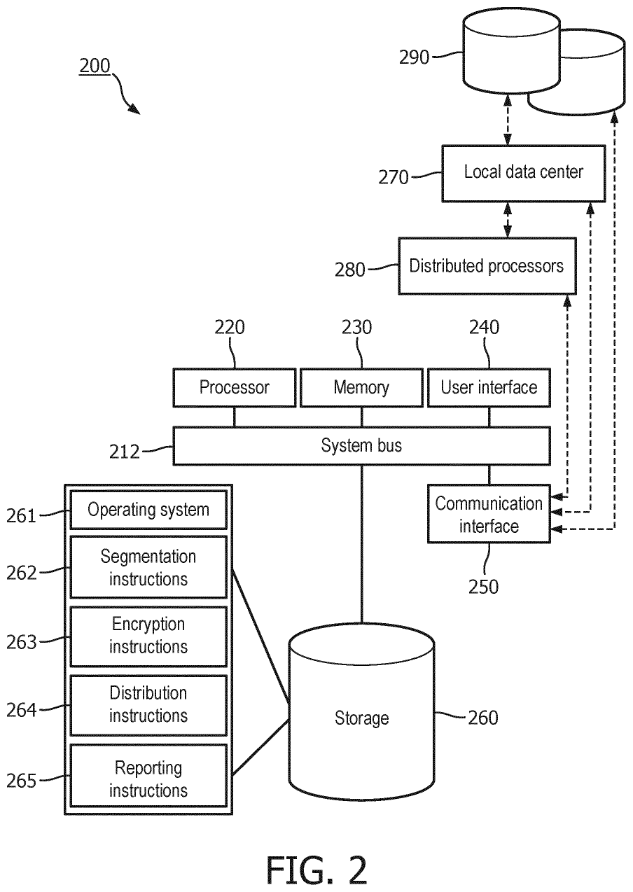

At step 110 of the method, a secure data processing system is provided. Referring to an embodiment of secure data processing system 200 as depicted in FIG. 2, for example, the system comprises one or more of a processor 220, memory 230, user interface 240, communications interface 250, and storage 260, interconnected via one or more system buses 212. The secure data processing system also optionally comprises a local data center 270, a plurality of distributed processors 280, and/or a plurality of distributed databases 290. It will be understood that FIG. 2 constitutes, in some respects, an abstraction and that the actual organization of the components of the system 200 may be different and more complex than illustrated. Additionally, secure data processing system 200 can be any of the systems described or otherwise envisioned herein. Other elements and components of the secure data processing system 200 are disclosed and/or envisioned elsewhere herein.

At step 120 of the method, the secure data processing system receives, retrieves, or otherwise obtains secure clinical data associated with a patient. The clinical data is secure in that it is sensitive data, such as patient data, and it is desired and/or required or mandated that the clinical data is protected such that only authorized individuals can access the clinical data. The clinical data can be any data, including but not limited to patient identification information, demographic information, diagnostic information, treatment information, clinical information such as medical data, imaging data, and/or any other information or data that is desired and/or required or mandated to be kept secure.

According to one non-limiting embodiment, the secure data processing system receives, retrieves, or otherwise obtains secure clinical data which represents imaging data from an imaging modality. The imaging modality may be ultrasound, X-ray, computer tomography (CT), magnetic resonance imaging (MRI), positron emission tomography (PET), and/or any other imaging modality. Thus, the secure clinical data may be imaging data obtained from any of these imaging modalities.

The secure clinical data can be received, retrieved, or otherwise obtained from one or a plurality of different sources. The secure clinical data can be received, retrieved, or otherwise obtained by the local data center 270 of the system, which may be some or many of the components of the system shown in FIG. 2. For example, the local data center may comprise everything other than the plurality of distributed processors 280 and the plurality of distributed databases 290, or may only comprise some of those components in FIG. 2 (in addition to components not shown). For example, the local data center may be a server, other computer, or other component located at a clinical setting such as an outpatient center, hospital, or doctor's office. As another example, the local data center may be the imaging device or system or other data-gathering device or system that gathers, creates, or otherwise generates the clinical data.

The received secure clinical data may be utilized immediately, or may be stored in local or remote storage for use in further steps of the method.

According to an embodiment, the secure clinical data may require processing after it is obtained. The processing may be any data processing that can be performed on clinical data, including for analytical or diagnostic purposes. The processing may be, for example, processing of imaging data to allow for analysis. Thus, also at step 120 of the method, the secure data processing system receives, retrieves, or otherwise obtains a processing request for the clinical data. The request may be an automated request, such that the processing of clinical data is part of an automated workflow. Alternatively, the request may be received via a user interface of the secure data system, from a clinician or other individual authorized to process or view the secure clinical data. The request may also be received from another system. The processing request can be any command for processing that can be performed on the received secure clinical data.

At step 130 of the method, the secure data processing system 200 determines, based on the received clinical data and/or the received processing request, a segmentation protocol for the received secure clinical data. The segmentation protocol may be any method, algorithm, or system for breaking down the received clinical data into two or more components. The segmentation protocol may be automatic such that it is part of an automated workflow, and thus the same segmentation protocol may be utilized for all clinical data and for every processing request. Alternatively, there may be two or more possible segmentation protocols that can be utilized and which of the two or more possible segmentation protocols is utilized depends upon either the nature of the received clinical data and/or on the demands of the received processing request. For example, a specific segmentation protocol may be identified for a specific imaging modality. The specific segmentation protocol may be predetermined or selected by a system setting, a parameter of the clinical data, or by a user. Thus, the segmentation protocol may be automatically determined or identified by the secure data processing system or may be manually determined or identified by a user, programmer, clinician, or other individual. Different segmentation protocols may differ in how they segment the data, how they package or process the data segments, or according to any other parameter. According to an embodiment, therefore, the secure data processing system 200 may comprise one or a plurality of segmentation protocols.

At step 140 of the method, the determined segmentation protocol is utilized to segment the received secure clinical data into a plurality of clinical data segments. The secure clinical data may be segmented by the segmentation protocol using any known method for data segmentation and/or packaging. Once the plurality of clinical data segments are generated by the determined segmentation protocol, the segments may be utilized immediately, or may be stored in local or remote storage for use in further steps of the method. According to an embodiment, the plurality of clinical data segments are segmented such that a clinical data segment sent to a distributed processor cannot identify the patient, or otherwise reveal sensitive information about the patient or about the clinical data.

At optional step 142 of the method, some or all of the plurality of clinical data segments may be encrypted. The clinical data segments can be encrypted using any known method, system, or algorithm for data encryption. Once encrypted, the segments may be utilized immediately, or may be stored in local or remote storage for use in further steps of the method. Notably, step 142 of the method may also occur before step 140 of the method.

At step 150 of the method, the plurality of clinical data segments are distributed to the plurality of distributed processors. The plurality of clinical data segments can be distributed to the plurality of distributed processors using any known method, system, or algorithm for distributing a data packet. For example, the data system may comprise an allocation algorithm or software service that packages or otherwise processes a data segment, and that then transmits the packaged data segment via a wired and/or wireless communication network to a distributed processor.

According to an embodiment, the distributed processor processes the clinical data segment. The processing may comprise any processing that the processor is designed or programmed or configured to perform. Each of the plurality of distributed processors that receives a clinical data segment may process the data immediately, or may store the clinical data segment for future processing. For example, a distributed processor may process data as it is received, and/or may batch process received data. Additionally, the distributed processor may return the processed data to the local data center immediately after it is processed, or it may store the processed data in local or remote storage until it is returned via the wired and/or wireless communication network to the local data center.

Thus, at step 160 of the method, the local data center of the data processing system receives the plurality of processed clinical data segments from the plurality of distributed processors. The processed clinical data segments can be received via the wired and/or wireless communication network using any method for receiving a data segment. This can be, for example, a function of the allocation algorithm of the data processing system. The received plurality of processed clinical data segments may be utilized immediately, or may be stored in local or remote storage for use in further steps of the method.

At step 170 of the method, the data processing system combines the received plurality of processed clinical data segments into a single combined processed secure clinical data result, effectively reversing the segmentation. The received plurality of processed clinical data segments can be combined into a single combined processed secure clinical data result using any method or algorithm for combining individual data packets. This may be performed by the segmentation protocol or algorithm, or may be performed by another protocol or algorithm and may optionally be based on the segmentation protocol or algorithm. Once the data processing system combines the received plurality of processed clinical data segments into a single combined processed secure clinical data result, that result may be utilized immediately or may be stored in local or remote storage for use in further steps of the method.

At optional step 180 of the method, the single combined processed secure clinical data result is provided via a user interface of the system. The single combined processed secure clinical data result may be provided to a user, which may be a clinician, patient, or medical professional, and/or to any other individual authorized to see or review the information. The single combined processed secure clinical data result can be provided to a user via any user interface or other information communication system. For example, the information can be transmitted or otherwise communicated via wired and/or wireless communication to a local or remote viewer, such as a remote medical professional.

At optional step 190 of the method, the single combined processed secure clinical data result can be utilized by a clinician or other medical professional to make a diagnosis and/or treatment recommendation for the patient. For example, the clinician or other medical professional can review the single combined processed secure clinical data result provided via the user interface, which can be a patient portal, imaging system, or other system or display or device. The clinician or other medical professional can utilize that displayed single combined processed secure clinical data result to make a diagnosis and/or treatment recommendation for the patient. Where the displayed single combined processed secure clinical data result is an image, for example, the clinician or other medical professional can review the image and make a diagnosis or treatment recommendation for the patient.

Accordingly, at step 192 of the method, the clinician or other medical professional can administer a treatment based on the diagnosis and/or treatment recommendation for the patient from step 190 of the method. Thus, the single combined processed secure clinical data result provided to the clinician or other medical professional via the user interface is implemented to make a diagnosis and/or treatment recommendation for the patient, and that treatment is then administered by the clinician or medical professional, or any other medical professional. The treatment can be any treatment that can be determined following analysis of clinical data. For example, analysis of a single processed image generated according to method 100 may determine that the patient has a broken bone, and the treatment is setting the bone and/or casting, among many other possible treatments.

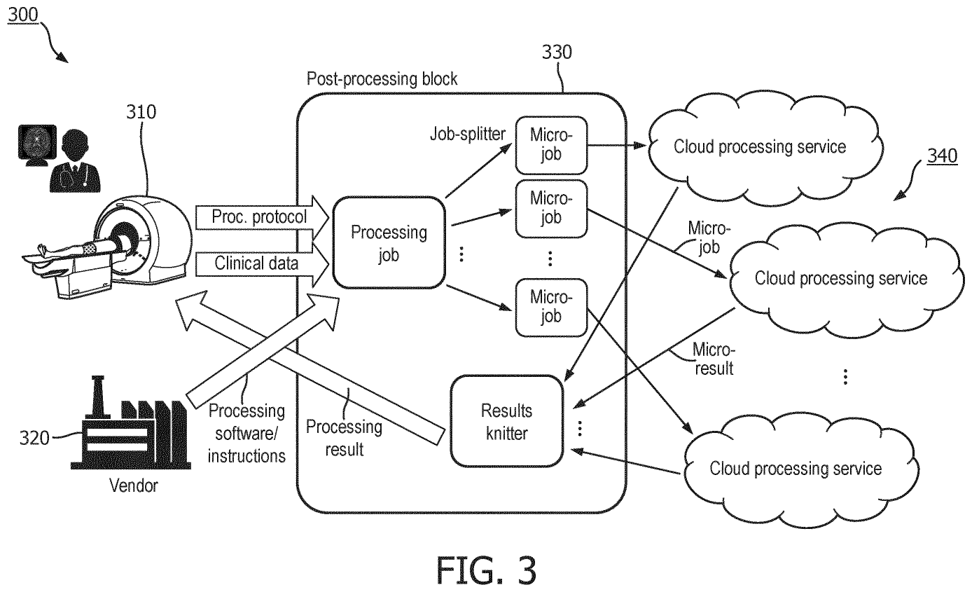

Referring to FIG. 3, in one embodiment, is a schematic representation of a system 300 for processing secure clinical data with a secure data processing system. For complex processing tasks, the processing job may consist of a dataset and a set of instructions or advanced description of the methods that should be applied to the dataset. For jobs that can run in parallel on several systems separately, each of these jobs are separated into micro-jobs which are sent to random remote processing instances which may be cloud-processing services run by various vendors and institutions. The instructions for each micro-job may be provided by the vendor, preferably in the most updated version. For AI-based methods, this could be the newest version of a trained network, or the newest software client version necessary to process the data.

Accordingly, a local data center 310 receives, retrieves, or otherwise obtains secure clinical data associated with a patient. The clinical data is secure in that it is sensitive data, such as patient data, and it is desired and/or required or mandated that the clinical data is protected such that only authorized individuals can access the clinical data. The clinical data can be any data, including but not limited to patient identification information, demographic information, diagnostic information, treatment information, clinical information such as medical data, imaging data, and/or any other information or data that is desired and/or required or mandated to be kept secure. According to one non-limiting embodiment, the secure data processing system receives, retrieves, or otherwise obtains secure clinical data which represents imaging data from an imaging modality. The local data center also receives, retrieves, or otherwise obtains a processing request for the clinical data. The request may be an automated request, such that the processing of clinical data is part of an automated workflow. Alternatively, the request may be received via a user interface of the secure data system, from a clinician or other individual authorized to process or view the secure clinical data. The request may also be received from another system. The processing request can be any command for processing that can be performed on the received secure clinical data.

As shown at 330, the local data center 310 receives the clinical data and the processing request or protocol, forming a processing job that must be performed. According to an embodiment, this processing job may be organized or processed or otherwise facilitated by processing software or instructions 320 provided or installed by a vendor. The local data center 310 segments the clinical data into a plurality of clinical data segments, which can each be called a “micro job” since each of the plurality of clinical data segments will be individually processed by a distributed processor as a micro job. The local data center 310 allocates or transmits the plurality of clinical data segments to different distributed remote processors, such as a cloud-based processing service, via a wired and/or wireless communication network. These distributed remote processors then perform the processing or micro job, to generate a “micro-result” which is returned to the local data center 310. The local data center 310 combines the micro-results to generate a single combined processed secure clinical data result (i.e., “processing result”), which can be provided to the clinician.

### EXAMPLE

The following is provided only as a non-limiting example of the processing of clinical data, and is therefore understood not to limit the scope of the embodiments described or otherwise envisioned herein.

The following example is an exemplary method or algorithm or process for distributed reconstruction of CT raw data. During CT data acquisition (such as a scout scan and 3D helical/axial scan), the raw data is split into N chunks (data segments) based on a selected CT protocol. The chunks must be large enough to allow independent reconstruction, but as small as possible for minimizing risk of identification and data misuse. According to an embodiment, keys which are known to the processing service are used for encryption of each data chunk. The encryption of a data chunk and the reconstruction settings from an encrypted mini-job, which is submitted or otherwise transmitted to a remote (cloud) processing service. The system waits and then receives the encrypted reconstructed results, which are decrypted, and then knitted or otherwise combined together to generate a full reconstructed data set. For a helical CT scan, for example, the raw data can be separated into overlapping data segments which can be reconstructed independently, each providing minimum diagnostic value on their own.

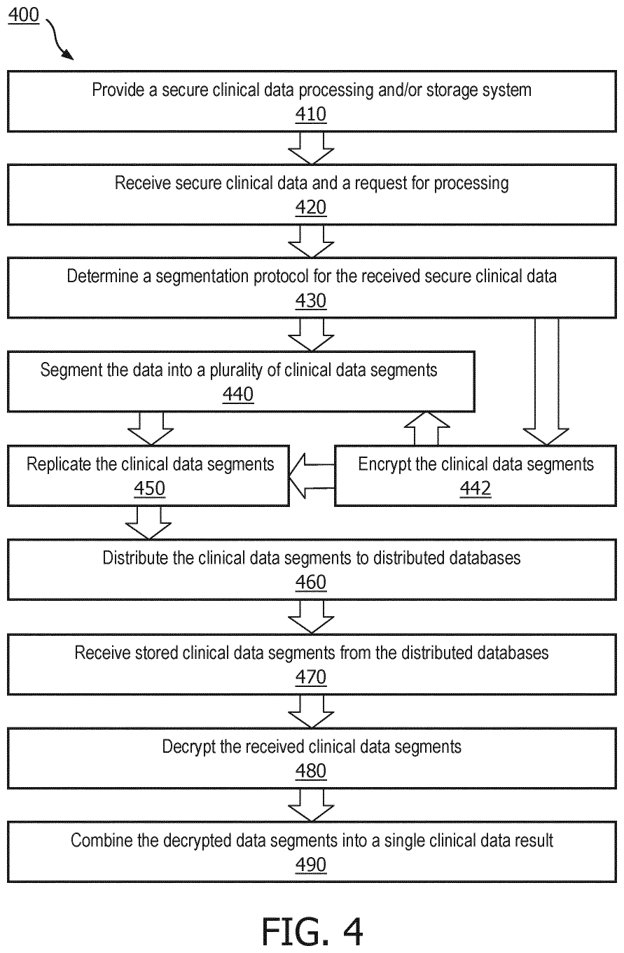

According to another embodiment, the secure data processing system is utilized to store secure clinical data. Referring to FIG. 4, in one embodiment, is a flowchart of a method 400 for storing secure clinical data by a secure data processing system. The methods described in connection with the figures are provided as examples only, and shall be understood not to limit the scope of the disclosure. The secure data processing system can be any of the systems described or otherwise envisioned herein. The secure data processing system can be a single system or multiple different systems.

At step 410 of the method, the secure data processing system is provided. Referring to an embodiment of secure data processing system 200 as depicted in FIG. 2, for example, the system comprises one or more of a processor 220, memory 230, user interface 240, communications interface 250, and storage 260, interconnected via one or more system buses 212. The secure data processing system also optionally comprises a local data center 270, a plurality of distributed processors 280, and/or a plurality of distributed databases 290. It will be understood that FIG. 2 constitutes, in some respects, an abstraction and that the actual organization of the components of the system 200 may be different and more complex than illustrated. Additionally, secure data processing system 200 can be any of the systems described or otherwise envisioned herein. Other elements and components of the secure data processing system 200 are disclosed and/or envisioned elsewhere herein.

At step 420 of the method, the secure data processing system receives, retrieves, or otherwise obtains secure clinical data associated with a patient. The clinical data is secure in that it is sensitive data, such as patient data, and it is desired and/or required or mandated that the clinical data is protected such that only authorized individuals can access the clinical data. The clinical data can be any data, including but not limited to patient identification information, demographic information, diagnostic information, treatment information, clinical information such as medical data, imaging data, and/or any other information or data that is desired and/or required or mandated to be kept secure.

According to one non-limiting embodiment, the secure data processing system receives, retrieves, or otherwise obtains secure clinical data which represents imaging data from an imaging modality. The imaging modality may be ultrasound, X-ray, computer tomography (CT), magnetic resonance imaging (MRI), positron emission tomography (PET), and/or any other imaging modality. Thus, the secure clinical data may be imaging data obtained from any of these imaging modalities.

The secure clinical data can be received, retrieved, or otherwise obtained from one or a plurality of different sources. The secure clinical data can be received, retrieved, or otherwise obtained by the local data center 270 of the system, which may be some or many of the components of the system shown in FIG. 2. For example, the local data center may comprise everything other than the plurality of distributed processors 280 and the plurality of distributed databases 290, or may only comprise some of those components in FIG. 2 (in addition to components not shown). For example, the local data center may be a server, other computer, or other component located at a clinical setting such as an outpatient center, hospital, or doctor's office. As another example, the local data center may be the imaging device or system or other data-gathering device or system that gathers, creates, or otherwise generates the clinical data. The received secure clinical data may be utilized immediately, or may be stored in local or remote storage for use in further steps of the method.

According to an embodiment, the secure clinical data may require storage after it is obtained. The storage may be temporary and/or permanent storage. Thus, also at step 120 of the method, the secure data processing system receives, retrieves, or otherwise obtains a storage request for the clinical data. The request may be an automated request, such that the storage of clinical data is part of an automated workflow. Alternatively, the request may be received via a user interface of the secure data system, from a clinician or other individual authorized to process or view the secure clinical data. The request may also be received from another system. The storage request can be any command for storage that can be performed on the received secure clinical data.

At step 430 of the method, the secure data processing system 200 determines, based on the received clinical data and/or the received storage request, a segmentation protocol for the received secure clinical data. The segmentation protocol may be any method, algorithm, or system for breaking down the received clinical data into two or more components. The segmentation protocol may be automatic such that it is part of an automated workflow, and thus the same segmentation protocol may be utilized for all clinical data and for every storage request. Alternatively, there may be two or more possible segmentation protocols that can be utilized and which of the two or more possible segmentation protocols is utilized depends upon either the nature of the received clinical data and/or on the demands of the received storage request. For example, a specific segmentation protocol may be identified for a specific imaging modality. The specific segmentation protocol may be predetermined or selected by a system setting, a parameter of the clinical data, or by a user. Thus, the segmentation protocol may be automatically determined or identified by the secure data processing system or may be manually determined or identified by a user, programmer, clinician, or other individual. Different segmentation protocols may differ in how they segment the data, how they package or process the data segments, or according to any other parameter. According to an embodiment, therefore, the secure data processing system 200 may comprise one or a plurality of segmentation protocols.

At step 440 of the method, the determined segmentation protocol is utilized to segment the received secure clinical data into a plurality of clinical data segments. The secure clinical data may be segmented by the segmentation protocol using any known method for data segmentation and/or packaging. Once the plurality of clinical data segments are generated by the determined segmentation protocol, the segments may be utilized immediately, or may be stored in local or remote storage for use in further steps of the method. According to an embodiment, the plurality of clinical data segments are segmented such that a clinical data segment sent to a distributed processor cannot identify the patient, or otherwise reveal sensitive information about the patient or about the clinical data.

At optional step 442 of the method, some or all of the plurality of clinical data segments may be encrypted. The clinical data segments can be encrypted using any known method, system, or algorithm for data encryption. Once encrypted, the segments may be utilized immediately, or may be stored in local or remote storage for use in further steps of the method. Notably, step 442 of the method may also occur before step 440 of the method.

At step 450 of the method, some or all of the plurality of encrypted clinical data segments are duplicated or replicated to generate redundancy of the secure clinical data. For example, the system may be programmed or designed to make one additional copy or multiple additional copies of the clinical data or of a clinical data segment. According to one embodiment, the duplicated clinical data segments may be distributed to different distributed databases in order to protect against data loss. The clinical data or clinical data segments may be duplicated or replicated using any known method for duplication or replication. The duplicated or replicated clinical data or clinical data segments may be utilized immediately or may be stored in local and/or remote storage for future use by the method.

Notably, step 450 of the method may be performed at any step of the method. For example, the clinical data may be duplicated before or after it is segmented, before or after it is encrypted, or before or after it is distributed or allocated to a distributed database.

At step 460 of the method, the plurality of encrypted clinical data segments are distributed to the plurality of distributed databases. The plurality of clinical data segments can be distributed to the plurality of distributed databases using any known method, system, or algorithm for distributing a data packet. For example, the data system may comprise an allocation algorithm or software service that packages or otherwise processes a data segment, and that then transmits the packaged data segment via a wired and/or wireless communication network to a distributed database. According to an embodiment, the distributed database stores the received clinical data segment(s).

At step 470 of the method, the local data center receives the plurality of encrypted clinical data segments from the plurality of distributed databases in response to a received request for the secure clinical data. For example, a clinician or other medical professional may request, via a user interface, clinical data that is remotely stored in the distributed databases. The local data center sends a request to the distributed databases to transmit the stored plurality of encrypted clinical data segments back to the local data center.

At step 480 of the method, the received plurality of encrypted clinical data segments are decrypted by the data processing system to generate a plurality of decrypted clinical data segments. The clinical data segments can be decrypted using any known method, system, or algorithm for data decryption. Once decrypted, the segments may be utilized immediately, or may be stored in local or remote storage for use in further steps of the method.

At step 490 of the method, the data processing system combines the plurality of decrypted clinical data segments to generate a single combined secure clinical data result, effectively reversing the segmentation. The plurality of decrypted clinical data segments can be combined into a single combined secure clinical data result using any method or algorithm for combining individual data packets. This may be performed by the segmentation protocol or algorithm, or may be performed by another protocol or algorithm and may optionally be based on the segmentation protocol or algorithm. Once the data processing system combines the received plurality of decrypted clinical data segments into a single combined secure clinical data result, that result may be utilized immediately or may be stored in local or remote storage for use in further steps of the method.

The single combined secure clinical data result can then be provided via a user interface of the system. The single combined secure clinical data result may be provided to a user, which may be a clinician, patient, or medical professional, and/or to any other individual authorized to see or review the information. The single combined secure clinical data result can be provided to a user via any user interface or other information communication system. For example, the information can be transmitted or otherwise communicated via wired and/or wireless communication to a local or remote viewer, such as a remote medical professional.

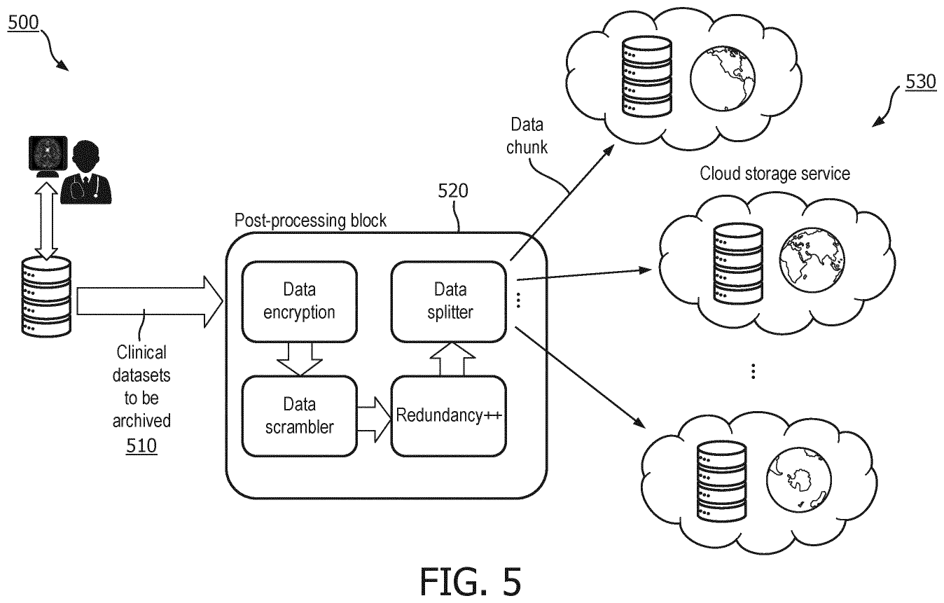

Referring to FIG. 5, in one embodiment, is a schematic representation of a system 500 for storing secure clinical data with a secure data processing system. For archiving a clinical dataset, the dataset is split into smaller chunks where each chunk does not contain sufficient information for data interpretation (such as patient identification or analysis). The dataset can be encrypted and scrambled before being split into smaller data chunks that are uploaded (outsourced) to cloud-based services in a redundant version. Redundancy is added so that if any data chunks are lost, the remaining distributed data allows reconstruction of the complete original clinical dataset. For this, the owning hospital must keep the links only to all data chunks together with critical patient and study information allowing to find the specific dataset and the location of its distributed data chunks.

Accordingly, at 510 the local data center receives a request to store data, such as a clinical dataset for archiving. At 520, the local data center encrypts the data, generates redundancy of the data, and segments the data into a plurality of encrypted clinical data segments. The plurality of encrypted clinical data segments are then distributed or allocated to a plurality of remote distributed databases 520, such as via a cloud-based storage service.

Although not shown in FIG. 5, the stored data can be restored. The local data center can transmit a request for the data to the plurality of remote distributed databases 520, which will return the plurality of encrypted clinical data segments to the local data center. The local data center then combines and decrypts the plurality of encrypted clinical data segments to generate a single combined secure clinical data result.

### EXAMPLE

The following is provided only as a non-limiting example of the storage of clinical data, and is therefore understood not to limit the scope of the embodiments described or otherwise envisioned herein.

For data storage, the data can be encrypted and scrambled. There can be RAID (redundant array of independent disks) transformation to generate (N+M) data chunks with specified redundancy level (i.e. N chunks are sufficient to unpack original data). Each data chunk or segment can be assigned a unique ID, and each can be transferred (and then locally deleted) to a remote cloud data storage service. That remote cloud data storage service cannot be the same for at least some N number of chunks, where N results in identification or other revelation of secure information. The system maintains a list of data chunk IDs and corresponding storage service identifiers saved on-site (e.g. in the clinic owning the data).

For data retrieval, the system can load the list of data chunk IDs and corresponding storage service identifiers saved on-site (e.g. in the clinic owning the data). The system can then submit a data retrieval request to each cloud service along with corresponding unique data chunk ID, and will retrieve at least N data chunks (out of total number N+M chunks). The system can then decrypt and combine the data chunks to generate a single recombined output, which can be saved or otherwise utilized.

Referring to FIG. 2 is a schematic representation of a secure data processing system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, the system comprises a local data center 270, which may be some or many of the components of the system shown in FIG. 2. For example, the local data center may comprise everything other than the plurality of distributed processors 280 and the plurality of distributed databases 290, or may only comprise some of those components in FIG. 2 (in addition to components not shown). For example, the local data center may be a server, other computer, or other component located at a clinical setting such as an outpatient center, hospital, or doctor's office. As another example, the local data center may be the imaging device or system or other data-gathering device or system that gathers, creates, or otherwise generates the clinical data.

According to an embodiment, the system comprises or is in communication with a plurality of distributed processors 280. According to an embodiment, the plurality of distributed processors are remote from the local data center. The plurality of distributed processors may be any processors, servers, computers, or other processing device that can process clinical data. For example, the plurality of distributed processors can be a remote service, such as a cloud-based service, that may or may not be hosted or provided by a third-party service or vendor.

According to an embodiment, the system comprises or is in communication with a plurality of distributed databases 290. According to an embodiment, the plurality of distributed databases are remote from the local data center. The plurality of distributed databases may be any memory, databases, computers, or other storage device that can store clinical data. For example, the plurality of distributed databases can be a remote service, such as a cloud-based service, that may or may not be hosted or provided by a third-party service or vendor.

According to an embodiment, storage 260 of system 200 may store one or more algorithms, modules, and/or instructions to carry out one or more functions or steps of the methods described or otherwise envisioned herein. For example, the system may comprise, among other instructions or data, segmentation instructions 262, encryption instructions 263, distribution instructions 264, and/or reporting instructions 265.

According to an embodiment, segmentation instructions 262 direct the secure data processing system to determine, based on the received clinical data and/or the received processing or storage request, a segmentation protocol for the received secure clinical data. The segmentation protocol may be any method, algorithm, or system for breaking down the received clinical data into two or more components. The segmentation protocol may be automatic such that it is part of an automated workflow, and thus the same segmentation protocol may be utilized for all clinical data and for every processing or storage request. Alternatively, there may be two or more possible segmentation protocols that can be utilized and which of the two or more possible segmentation protocols is utilized depends upon either the nature of the received clinical data and/or on the demands of the received processing or storage request. For example, a specific segmentation protocol may be identified for a specific imaging modality. The specific segmentation protocol may be predetermined or selected by a system setting, a parameter of the clinical data, or by a user. Thus, the segmentation protocol may be automatically determined or identified by the secure data processing system or may be manually determined or identified by a user, programmer, clinician, or other individual.

According to an embodiment, the segmentation instructions 262 also direct the secure data processing system to segment the received secure clinical data into a plurality of clinical data segments using any known method for data segmentation and/or packaging. Once the plurality of clinical data segments are generated by the determined segmentation protocol, the segments may be utilized immediately, or may be stored in local or remote storage for use in further steps of the method. According to an embodiment, the plurality of clinical data segments are segmented such that a clinical data segment sent to a distributed processor cannot identify the patient, or otherwise reveal sensitive information about the patient or about the clinical data.

According to an embodiment, the segmentation instructions 262 also direct the secure data processing system to combine processed clinical data segments into a single combined processed secure clinical data result, effectively reversing the segmentation, The received plurality of processed clinical data segments can be combined into a single combined processed secure clinical data result using any method or algorithm for combining individual data packets.

According to an embodiment, the segmentation instructions 262 also direct the secure data processing system to combine decrypted clinical data segments to generate a single combined secure clinical data result, effectively reversing the segmentation. The plurality of decrypted clinical data segments can be combined into a single combined secure clinical data result using any method or algorithm for combining individual data packets.

According to an embodiment, encryption instructions 263 direct the secure data processing system to encrypt some or all of the plurality of clinical data segments before processing or storage. The clinical data segments can be encrypted using any known method, system, or algorithm for data encryption. Once encrypted, the segments may be utilized immediately, or may be stored in local or remote storage for use in further steps of the method.

According to an embodiment, encryption instructions 263 direct the secure data processing system to decrypt some or all of the plurality of clinical data segments after processing or storage. The clinical data segments can be decrypted using any known method, system, or algorithm for data decryption. Once decrypted, the segments may be utilized immediately, or may be stored in local or remote storage for use in further steps of the method.

According to an embodiment, distribution instructions 264 direct the secure data processing system to distribute or allocate the plurality of clinical data segments to the plurality of distributed processors or the plurality of distributed databases. The plurality of clinical data segments can be distributed to the plurality of distributed processors or the plurality of distributed databases using any known method, system, or algorithm for distributing a data packet. For example, the data system may comprise an allocation algorithm or software service that packages or otherwise processes a data segment, and that then transmits the packaged data segment via a wired and/or wireless communication network to a distributed processor or database.

According to an embodiment, distribution instructions 264 also direct the secure data processing system to request the processed or stored plurality of clinical data segments from the plurality of distributed processors or the plurality of distributed databases, such as in response to a request for the data via a user interface or an automated workflow.

According to an embodiment, reporting instructions 265 direct the secure data processing system to provide, via a user interface, the single combined processed secure clinical data result or the single combined secure clinical data result. The result may be provided to a user, which may be a clinician, patient, or medical professional, and/or to any other individual authorized to see or review the information. The result can be provided to a user via any user interface or other information communication system. For example, the information can be transmitted or otherwise communicated via wired and/or wireless communication to a local or remote viewer, such as a remote medical professional.

According to an embodiment, the secure data processing system 200 is configured to process many thousands or millions of datapoints from the input to the system (i.e., the received clinical data) in order to generate the output (i.e., the plurality of clinical data segments and/or the single combined processed secure clinical data result or the single combined secure clinical data result). Processing this data in a secure way-to avoid patient identification or the analysis of secure data-comprises a process with a volume of calculation and analysis that a human brain cannot accomplish in a lifetime, or multiple lifetimes. Indeed, one of the benefits of a computer-based system is that a human that might perform data segmentation would inadvertently see the data prior to segmentation, meaning that segmentation would not ensure the security of the clinical data.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for processing secure clinical data the method comprising:
distributing a plurality of clinical data segments to a plurality of distributed processors wherein the clinical data segments are segmented based on a segmentation protocol;
processing the distributed plurality of clinical data segments by the plurality of distributed processors to generate a plurality of processed clinical data segments; and
combining the plurality of the processed clinical data segments to generate a single combined processed clinical data result.

2. The method of claim 1, wherein the clinical data is imaging data obtained by an imaging modality, and wherein the imaging modality is a local data center or is in communication with the local data center.

3. The method of claim 1, further comprising encrypting at least a portion of the plurality of clinical data segments.

4. The method of claim 1, wherein the plurality of clinical data segments are segmented such that a clinical data segment sent to a distributed processor cannot identify the patient.

5. The method of claim 1, further comprising providing, via a user interface, the single combined processed secure clinical data result.

6. The method of claim 1, further comprising:
diagnosing the patient based on the single combined processed secure clinical data result; and
administering a treatment based on the diagnosis.

7. A non-transitory machine readable medium comprising instructions for processing secure clinical data in accordance with a segmentation protocol, the instructions, when executed by a processor, cause the processor to:
distribute a plurality of clinical data segments to a plurality distributed processors for processing the plurality of clinical data segments to generate a plurality of processed clinical data segments, wherein the clinical data segments are segmented based on the segmentation protocol; and
combine the plurality of the processed clinical data segments to generate a single combined processed clinical data result.

8. The non-transitory machine readable medium of claim 7, wherein the instructions, when executed by the processor, further cause the processor to encrypt at least a portion of the plurality of clinical data segments.

9. The non-transitory machine readable medium of claim 8, the instructions, when executed by the processor, further cause the processor to:
receive a plurality of encrypted processed clinical data segments from the plurality of distributed databases;
decrypt the received plurality of encrypted processed clinical data segments to generate a plurality of decrypted processed clinical data segments; and
combine the plurality of decrypted processed clinical data segments to generate a single combined secure clinical data result.

10. The non-transitory machine readable medium of claim 9, wherein a local data center maintains a record of which distributed databases store which clinical data segments.

11. The non-transitory machine readable medium of claim 7, wherein the instructions, when executed by the processor, further cause the processor to replicate at least a portion of the plurality of clinical data segments.

12. The non-transitory machine readable medium of claim 7, wherein the instructions, when executed by the processor, further cause the processor to provide, via a user interface, the single combined secure clinical data result.

13. A secure data processing system for processing secure clinical data in accordance with a segmentation protocol, the system comprising:
a processor configured to:
distribute a plurality of clinical data segments to a plurality distributed processors for processing the plurality of clinical data segments to generate a plurality of processed clinical data segments, wherein the clinical data segments are segmented based on the segmentation protocol; and
combine the plurality of the processed clinical data segments to generate a single combined processed clinical data result.

14. The secure data processing system of claim 13, wherein the processor is further configured to encrypt at least a portion of the plurality of clinical data segments.

15. The secure data processing system of claim 13, wherein the processor is further configured to provide, via a user interface, the single combined secure clinical data result.

16. The secure data processing system of claim 13, wherein the processor is further configured to replicate at least a portion of the plurality of encrypted clinical data segments.

17. The secure data processing system of claim 14, wherein the processor is further configured to: (v) receive a plurality of encrypted processed clinical data segments from the plurality of distributed processors; (vi) decrypt the received plurality of encrypted processed clinical data segments to generate a plurality of decrypted processed clinical data segments; and (vii) combine the plurality of decrypted clinical data segments to generate a single combined secure clinical data result.
